(19) United States
(12) Patent Application Publication
(10) Pub. No.: US 2020/0038594 A1
(43) Pub. Date: Feb. 6, 2020

(54) DRUG DELIVERY SYSTEM

(71) Applicant: JASON COOK, ARDEN, NC (US)

(72) Inventor: JASON COOK, ARDEN, NC (US)

(21) Appl. No.: 16/533,391

(22) Filed: Aug. 6, 2019

Related U.S. Application Data
(60) Provisional application No. 62/714,800, filed on Aug. 6, 2018.

(51) Int. Cl. A61M 5/315; A61M 15/08

(52) U.S. Cl. CPC A61M 5/3159; A61M 11/06; A61M 15/08

(57) ABSTRACT

A delivery system for intranasally administering doses of a substance such as a drug, vaccine, or the like is provided. The drug delivery system includes a holder comprising a rear case and a pre-filled drug container that is securely connected to the holder. The drug container includes a cartridge for containing the drug, a movable stopper within the cartridge, and an end having an opening through which the substance within the cartridge can be expelled. The rear case is connected to the drug container at one end and an activator at the other end. Within the rear case is contained the means for activating the movable stopper within the cartridge, which acts as a plunger rod, by pressing the activator. Further provided is a nozzle able to be attached to the opening at the end of the drug container to provide a means for precise delivery of the drug.

This application claims priority from the provisional application, U.S. Application Ser. No. 62/714,800, filed Aug. 6, 2018.

### FIELD OF THE INVENTION

The present invention generally relates to delivery systems for delivering substances such as drugs, vaccines, and the like and more specifically relates to a drug delivery system for preferably delivering such substances intranasally, i.e., through the nose. The system includes a prefilled drug cartridge with a movable stopper within, a means for activating the movable stopper, and a nozzle attached to the cartridge to allow for precise delivery of the drug. The inventive system is sized for use by one hand.

### BACKGROUND OF THE INVENTION

A number of medications may be effectively administered through the nasal passages. Devices have accordingly been developed for this purpose including either cartridges, such as those described in U.S. Pat. No. 6,622,721 (Vedrine et al.), U.S. Pat. No. 5,893,484 (Fuchs et al.), U.S. Pat. No. 5,813,570 (Fuchs et al.), U.S. Pat. No. 5,655,689 (Fuchs et al.), U.S. Pat. No. 5,511,698 (Solignac), U.S. Pat. No. 5,427,280 (Fuchs), U.S. Pat. No. 5,289,818 (Citterio et al.), U.S. Pat. No. 5,284,132 (Geier) and U.S. Pat. No. 5,171,219 (Fujioka et al.), or syringes, such as those described in U.S. Pat. No. 5,601,077 (Imbert), U.S. Pat. No. 4,923,448 (Ennis, III), U.S. Pat. No. 4,767,416 (Wolf et al.) and U.S. Pat. No. 4,344,573 (De Felice).

The nasal syringes are usually of a more conventional construction such as that described in U.S. Pat. No. 5,601,077 (Imbert) which includes a cylindrical cartridge having a blunt tip portion for insertion into a nostril. A stopper is positioned within the cartridge. A plunger extends from the end of the cartridge opposite to the blunt tip. The plunger controls the position of the stopper within the cartridge. A flange may be provided on one end of the plunger to facilitate its use. However, limitations remain, particularly with respect to ensuring that sufficient force is applied to obtain effective application.

Nasal syringes are often supplied to users pre-filled with medication. Whether pre-filled or not, it may be desirable to administer selected, and usually equal volumes of medication to each nostril. U.S. Pat. No. 4,962,868 (Borchard) discloses the use of a telescoping tube assembly which is designed for expelling the contents of a nasal syringe in two controlled doses. Also, U.S. Pat. No. 5,601,077 (Imbert) discloses the use of a dose limited in the form of a c-shaped attachment on the plunger rod for limiting movement in the distal directed so that approximately half of the substance to be delivered remains in the syringe. To continue, the user simply removes the attachment from the plunger rod. In addition, U.S. Pat. No. 5,951,526 (Korisch et al.) discloses a holder having an integral dose divider. However, it remains difficult for users to administer equal doses of medication to each nostril, especially with one hand.

U.S. Pat. No. 6,622,721 (Vedrine et al.) discloses a nasal drug delivery system which is able to deliver a substance, especially intranasally, easily with one hand, including delivery of a uniform spray which can be divided into at least two separate doses for delivery into each nostril. However, it remains difficult to provide precise delivery of a viscous liquid such as, for example, to the sphenopalatine ganglion (SPG).

Conventional methods for treating pain associated with headaches and facial aches are not as safe or effective as desired. By way of example, non-steroidal anti-inflammatory drugs (NSAIDs), such as the COX-2 brand of medications, must be used sparingly and only for short durations in view of their potential for causing ulcers and heart attacks—a drawback that is further compounded by the inefficacy of these medications in a large number of patients. The use of narcotics is likewise undesirable in view of their potentially addictive properties. In addition, the use of tryptamine-based drugs which include but are not limited to sumatriptan (sold under the tradename IMITREX by GlaxoSmithKline) and zolmitriptan (sold under the tradename ZOMIG by AstroZeneca)—is undesirable in view of the costliness and potentially high toxicity of these drugs.

It is well known that the application of a local anesthetic, such as lidocaine, to the sphenopalatine ganglion (SPG) can be effective in reducing the pain associated with migraines. This has been historically accomplished through the use of a cotton swab applicator saturated in viscous local anesthetic. However, this method requires the assistance or supervision of a trained medical professional. More recent innovations as described in U.S. Pat. Nos. 8,231,888 and 8,905,980 (Xia) provide for spraying such medication by using a nasal applicator. These devices do not provide guidance regarding the depth of insertion into the nasal passageway without assistance. Moreover, these Xia devices are not directed for use with viscous liquids since they are designed to atomize medication, which requires the use of liquids with lower viscosity.

Accordingly, there has been a need for a device that allows a user to attain precise delivery of a viscous liquid without supervision or assistance from a medical professional.

### SUMMARY OF THE INVENTION

In contrast to the prior devices discussed above, it has been found that a drug delivery system particularly suited for use in intranasally delivering substances such as drugs, vaccines and the like can be constructed in accordance with the present invention. Specifically, the invention is directed to an assembly such as a housing which allows the use of a pre-filled drug containers while providing control of the dose to be administered and further providing precise delivery of a viscous liquid. The assembly is particularly applicable to nasal syringes where it may be desirable to dispense medication in two equal doses.

The system of the present invention for delivering at least one substance in at least one dose includes a drug container including a cartridge having a first end extending from the cartridge and a stopper slidably positioned within the cartridge, a holder having a distal portion and a proximal portion with the distal portion being assembled to the proximal portion and the drug container secured therein, and a means for controlling the delivery of a substance contained in the cartridge of the drug container whereby upon activation of the system, the portions of the holder move towards one another upon the application of a minimum force and the stopper moves to expel at least a portion of the substance from the drug container.

The system further includes a nozzle for use in intranasally delivering the substance which is able to be attached to a second end of the cartridge and provides for precise delivery of a drug.

The system may further include a drug container capable of holding at least two doses of the drug and a means for controlling the dispensation of the drug such that each dose is separately administered.

### BRIEF DESCRIPTION OF THE DRAWINGS

The various features, objects, benefits, and advantages of the present invention will become more apparent upon reading the following detailed description of the preferred embodiment along with the appended claims in conjunction with the drawings, wherein like reference numerals identify corresponding components, and:

FIG. 1 is a perspective view of an embodiment of the drug delivery system of the present invention.

FIG. 2 is a perspective view of an embodiment of the drug delivery system of the present invention.

FIG. 3 is a side view of the drug delivery system of an embodiment of the present invention as it might be inserted into a user with the user being shown in cross-section to demonstrate the insertion.

### DETAILED DESCRIPTION OF THE DRAWINGS

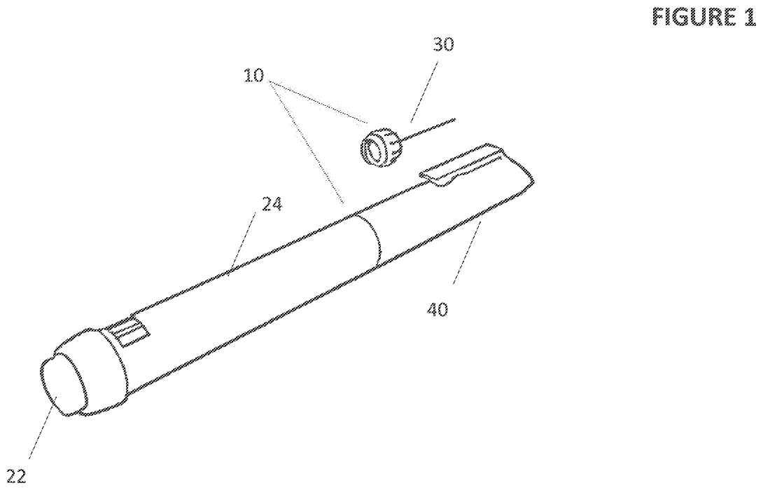

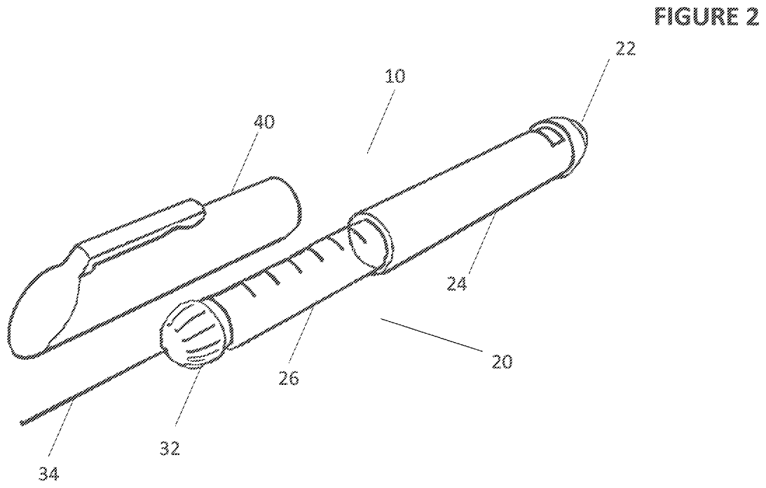

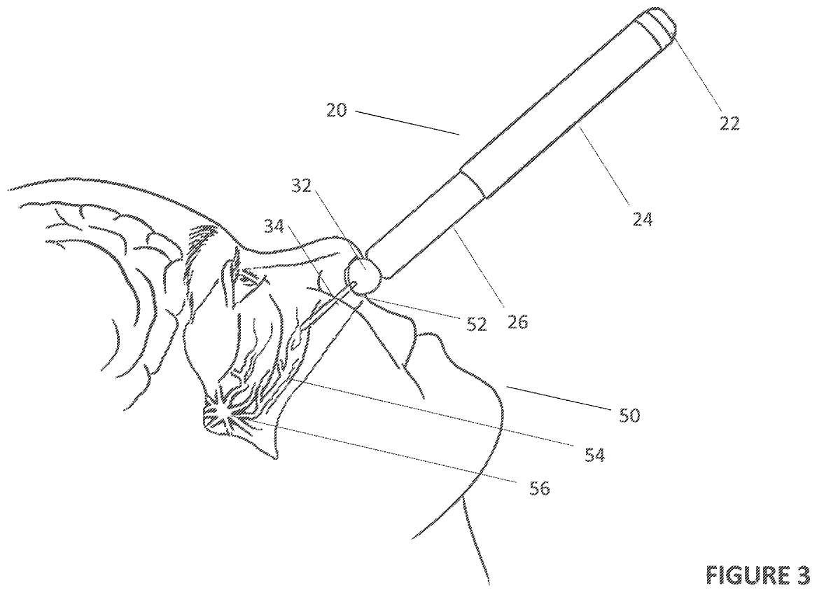

The drug delivery system of the present invention is illustrated in FIGS. 1-3 and generally includes the designation 10. Referring to FIGS. 1 and 2, the system 10 of the present invention includes a holder 20, a nozzle 30, and a cap 40. When used to deliver substances intranasally, as shown in FIG. 3, the system 10 ensures that the substance to be delivered by the syringe is applied within the pterygopalatine fossa 54 such that it will reach the sphenopalatine ganglion (SPG) 56. The design of the nozzle 30 limits the penetration of the shaft 34 of the nozzle 30 such that the distal end of the shaft 34 will reach an appropriate place within or near to the pterygopalatine fossa 54 and no further.

Referring to FIGS. 1 and 2, the holder 20 of the system 10 is comprised of at least two portions, a rear case section 24 at the distal end and, connected to it, a cartridge 26 at the proximal end. For the purposes of the description of the present invention, the term distal is meant to refer to the portion or end furthest from the person holding the system 20 of the present invention and the term proximal is meant to refer to the portion or end closest to the person holding the system 20.

The rear case section 24 and a cartridge 26 may have a generally tubular interior configuration as shown or some other shape that is convenient for the containing and application of a drug that may be contained within the cartridge 26. Contained within the rear case section 24 is a mechanism (not shown) for activating a stopper (not shown) within the cartridge 26. At the distal end of the rear case section 24 is an activator 22 which may be used to trigger the mechanism for activating the stopper.

The rear case section 24 may further include a means for activating the stopper such that only a portion of the drug is dispelled. This means for delivering only a portion of the drug contained in the cartridge 26 may incorporate a dosage selector dial (not shown).

The cartridge 26 is connected at its distal end to the rear case section 24 such that the stopper within the cartridge 26 may be activated by the mechanism contained in the rear case section 24. At its proximal end is a means for containing the drug without leakage. This may be, for example, a removable tip cap or a flow control valve. Additionally, the proximal end of the cartridge 26 includes a means for connecting the nozzle 30 to the system 10. The cartridge 26 may be clear and further may have volume indicator marks to allow a user 50 to observe the volume of drugs contained within the cartridge 26.

The cartridge 26 may be removably connected to the rear case section 24 such that the user 50 will be able to obtain new cartridges 26 and the rear case section 24 may be used repeatedly.

The nozzle 30 comprises a bulb 32 and a shaft 34, both of which are substantially hollow on the interior. The distal end of the nozzle 30 is designed to securely connect to the proximal end of the cartridge 26. The connection between the two parts may be through a threaded fit (shown in FIG. 1), a pressure fit, or a twist lock. The bulb 32 is sized such that, as shown in FIG. 3, it fits to the nostril 52 in such a way as to guide the user 50 in positioning the shaft 34 within the nasal passage (not shown). The shaft 34 is very small in exterior diameter such that it will easily fit within the nasal passage, such as, for example, about 2 mm. The length of the shaft 34 will range from 1.5 cm to 3.5 cm and may even be customizable in length with guidance from a medical professional. The tip of the shaft 34 should be slightly rounded or soft at the end and should contain one or a plurality of apertures configured for dispersing the drug. Both the bulb 32 and the shaft 34 should be manufactures of flexible material, though they need not be made of the same material. It is most important that the bulb 32 be sufficiently soft that, during insertion, it is able to curve within the nasal passage of the user 50 and will not damage or irritate the nasal passage of the user 50.

A method for ameliorating pain in a patient in accordance with the present teachings includes delivering a local anesthetic towards the SPG 56 of a user 50 by using a system 10 as described herein. In some embodiments, the anesthetic is delivered laterally and/or superiorly towards the SPG 56. In other embodiments, the anesthetic is delivered laterally, superiorly, and anteriorly towards the SPG 56. In some embodiments, a method for ameliorating pain in a user 50 includes (a) introducing the shaft 34 of the nozzle 30 into the nostril 52 of a user 50 and through a nasal passage of the patient until the bulb 32 of the nozzle 30 comes into gentle contact with the nostril 52 and allowing the shaft 34 to reach into a region substantially medial and/or posterior and/or inferior to an SPG 56 of the user 50; and (b) delivering anesthetic from the cartridge 26 and through the nozzle 30 superiorly and/or laterally and/or anteriorly towards the SPG 56.

Anesthetics that may be used in accordance with embodiments described herein include but are not limited to ambucaine, amolanone, amylocalne, benoxinate, betoxycaine, biphenamine, bupivacaine, butacaine, butamben, butanilicicaine, butethamine, butoxycaine, carticaine, cocaethylene, cocaine, cyclomethycaine, dibucaine, dimethisoquin, dimethocaine, diperodon, dyclonine, ecgonidine, ecgonine, ethyl aminobenzoate, ethyl chloride, etidocaine, β-eucaine, euprocin, fenalcomine, fomocaine, hexylcaine, hydroxyprocaine, hydroxytetracaine, isobutyl p-aminobenzoate, leucinocaine mesylate, levoxadrol, lidocaine, meperidine, mepivacaine, meprylcaine, metabutoxycaine, methyl chloride, myrtecaine, naepaine, octacaine, orthocaine, oxethazaine, parethoxycaine, phenacaine, phenol, a pipecoloxylidide, piperocaine, piridocaine, polidocanol, pramoxine, sameridine, prilocalne, propanocaine, proparacaine, propipocaine, propoxycaine, pseudococaine, pyrrocaine, quinine urea, risocaine, ropivacaine, salicyl alcohol, tetracaine, tolycaine, trimecaine, veratridine, zolamine, and the like, and combinations thereof, as well as all optical and/or stereoisomers thereof, and all pharmaceutically acceptable salts thereof.

In some embodiments, the drug comprises an anesthetic selected from the group consisting of benzocaine, tetracaine, ropivacaine, lidocaine, water, saline, and combinations thereof. In some embodiments, the drug comprises water and/or saline having a temperature of less than about 10° C. and in other embodiments of less than about 5° C. In some embodiments, the drug comprises water and/or saline having a temperature of about 4° C. In some embodiments, the drug comprises a combination of benzocaine, tetracaine, and ropivacaine. In some embodiments, the drug comprises an anesthetic comprising about 14% benzocaine, about 2% tetracaine, and about 1% ropivacaine by weight based on total weight of the anesthetic.

In some embodiments, a mixture of benzocaine, tetracaine, and ropivacaine is used to achieve a fast onset of SPG block as well as to prolong the effects of pain relief, thereby reducing the need for repeated applications and minimizing any potential dose-related complications and/or side effects. Benzocaine—which is quite effective in topical use and has a toxic dose in excess of about 200 mg—has an onset time of about 30 seconds and lasts for between about 0.5 and about 1 hour. Benzocaine provides an almost immediate onset of pain relief and may increase the absorption of other local anesthetics when mixed therewith. Ropivacaine—which has a toxic dose of about 175 mg—typically has a slow onset but lasts for between about 2 and about 6 hours. Ropivacaine provides an extended nerve block and lasting pain relief. Tetracaine is a very intense local anesthetic having a fast onset and lasting for between about 0.5 and about 1 hour. When tetracaine is combined with ropivacaine, the duration of pain relief exceeds 6 hours.

The amount of drug delivered in accordance with the present teachings can be readily determined by one of ordinary skill in the art and will vary according to factors such as the nature and/or concentration of the drug, the patient's age, condition, and/or sensitivity to the drug, and the like. In some embodiments, the dosage of anesthetic ranges from about 0.1 cc to about 1.0 cc. In some embodiments, the dosage of anesthetic is about 0.5 cc.

Methods and devices described herein are contemplated for use in the treatment of all manner of conditions for which the introduction of a drug superiorly and/or laterally and/or anteriorly towards the SPG of a patient is desirable. Representative conditions that can be treated include but are not limited to sphenopalatine neuralgia, trigeminal neuralgia including glossopharyngeal neuralgia, migraine with or without aura, tension headaches, cluster headaches including chronic cluster headaches, paroxysmal hemicranias, superior laryngeal neuralgia, atypical facial pain, herpes zoster opthalmicus, vasomotor rhinitis, major depression, fibromyalgia, and the like, and combinations thereof.

Topical administrations of a drug to human tissue for the systemic delivery of a pharmaceutically active agent typically include the use of transdermal and/or transmucosal pastes, creams, liquids, solids, semisolids, and the like. However, systemic delivery of pharmaceutically active agents by topical administration is hampered by the difficulty of diffusing an agent through the tissue to which the agent is applied in order to reach blood vessels, whereby the agent can then be absorbed for systemic delivery. Thus, to address this difficulty, the methods and devices described herein may be invoked to achieve increased permeability of the blood brain barrier in the administration of any drug.

Conventional SPG block procedures have been used to treat a wide array of patient ailments, and the methods and devices described herein are contemplated for use in the treatment of all of them. Representative ailments include but are not limited to the pain and/or discomfort associated with muscle spasm, vascospasm, neuralgia, reflex sympathetic dystrophy, chronic low back pain of multiple etiology (e.g., muscular, discogenic, arthritic, etc.), external cricoidynia, lower jaw toothache, glossodynia, earache (in case of Eustachian tube) and middle ear lesions, earache secondary to cancer of the larynx, pain from laryngeal tuberculosis, spasm of the face and upper respiratory tract, syphilitic headache, malarial headache, cluster headache, ophthalmic migraine, dysmenorrheal, intercostal pain (neuralgia), gastric pain, nausea and diarrhea, myalgias of the neck muscles, sciatica, maxillary neuralgia, sensory facial neuralgia, upper teeth pain, pain associated with tooth extraction, feeling of foreign body in the throat, persistent itching in the external ear canal, herpes zoster oticus, taste disturbances, atypical facial pain, tic douloureux, cervical arthritis, myofascial syndrome, peripheral neuropathy, post-herpetic neuralgia, fracture secondary to osteoporosis, lumbosacral strain, extremity arthritis, various other arthritic conditions, and the like, and combinations thereof. Further indications for which the devices and methods described herein are contemplated include but are not limited to rage control, depression, amelioration, and the like.

The foregoing detailed description and accompanying drawings have been provided by way of explanation and illustration, and are not intended to limit the scope of the appended claims. Many variations in the presently preferred embodiments illustrated herein will be apparent to one of ordinary skill in the art, and remain within the scope of the appended claims and their equivalents.

The invention claimed is:

1. A device for intranasally delivering a drug to a user in need thereof comprising:
a. a cartridge for holding a drug having a distal end with a first connecting means, having a proximal end with a second connecting means, and containing within a stopper,
b. a rear case section having an operating means for activating the stopper within the cartridge, having a proximal end with a third connecting means for connecting to the second connecting means of the cartridge, and having a distal end comprising an activator means for activating the operating means, and
c. a nozzle being hollow and having a distal end with a fourth connecting means for connecting to the first connecting means of the cartridge and allowing passage of the drug from the cartridge into the nozzle, a bulb at the distal end of the nozzle for fitting against the user's nostril, and a shaft connected to the bulb and extending into the user's nasal passage with a length sufficient for the shaft to deliver through an aperture configured for dispersing the drug toward a user's sphenopalatine ganglion.

2. The device of claim 1 wherein the shaft of the nozzle further comprises a plurality of apertures for dispersing the drug toward a user's sphenopalatine ganglion.

3. The device of claim 1 wherein the shaft of the nozzle is configured to deliver the drug towards the user's sphenopalatine ganglion at least one of laterally, superiorly, anteriorly, and any combination of laterally, superiorly, or anteriorly.

4. The device of claim 1 wherein the shaft of the nozzle is contoured such that it is configured to be complementary in shape to an interior of a human nostril.

5. The device of claim 1 wherein the rear case section further comprises a means for selecting a dosage amount of the drug.

6. The device of claim 1 wherein the cartridge configured to contain at least two doses of the drug.

7. The device of claim 6 wherein the operating means for activating the stopper within the cartridge further comprises a means for delivering a first half of the drug contained in the cartridge.

8. The device of claim 7 wherein the operating means for activating the stopper within the cartridge further comprises a means for delivering a second half of the drug contained in the cartridge.

9. The device of claim 1 wherein the cartridge further comprises a removable tip cap at the cartridge's proximal end.

10. The device of claim 1 wherein the cartridge further comprises a flow control valve at the cartridge's proximal end.

11. The device of claim 1 wherein the shaft of the nozzle is able to be sized in a custom manner.

12. The device of claim 1 wherein the activator means is positioned such that the user may activate the activator means by use of a thumb when grasping the device in a hand.

13. The device of claim 11 wherein the activator means is directed toward the user.

14. A method for using the device of claim 1 to deliver a dose of the drug to the user comprising: introducing the shaft of the nozzle into the user's first nasal passage, engaging the bulb with the first nostril of the user, activating the activator means, and delivering the drug towards the user's sphenopalatine ganglion from the cartridge through the nozzle.

15. The method of claim 14 wherein the device of claim 1 is further used to deliver a second dose of the drug to the user comprising: comprising: introducing the shaft of the nozzle into the user's second nasal passage, engaging the bulb with the second nostril of the user, activating the activator means, and delivering the drug towards the user's sphenopalatine ganglion from the cartridge through the nozzle.

16. The method of claim 14 wherein the drug is delivered towards the user's sphenopalatine ganglion at least one of laterally, superiorly, anteriorly, and any combination of laterally, superiorly, or anteriorly.

17. The method of claim 14 wherein the drug is selected from a group comprising ambucaine, amolanone, amylocalne, benoxinate, betoxycaine, biphenamine, bupivacaine, butacaine, butamben, butanilicicaine, butethamine, butoxycaine, carticaine, cocaethylene, cocaine, cyclomethycaine, dibucaine, dimethisoquin, dimethocaine, diperodon, dyclonine, ecgonidine, ecgonine, ethyl aminobenzoate, ethyl chloride, etidocaine, β-eucaine, euprocin, fenalcomine, fomocaine, hexylcaine, hydroxyprocaine, hydroxytetracaine, isobutyl p-aminobenzoate, leucinocaine mesylate, levoxadrol, lidocaine, meperidine, mepivacaine, meprylcaine, metabutoxycaine, methyl chloride, myrtecaine, naepaine, octacaine, orthocaine, oxethazaine, parethoxycaine, phenacaine, phenol, a pipecoloxylidide, piperocaine, piridocaine, polidocanol, pramoxine, sameridine, prilocalne, propanocaine, proparacaine, propipocaine, propoxycaine, pseudococaine, pyrrocaine, quinine urea, risocaine, ropivacaine, salicyl alcohol, tetracaine, tolycaine, trimecaine, veratridine, zolamine, and combinations thereof, as well as all optical and/or stereoisomers thereof, and all pharmaceutically acceptable salts thereof.

18. The method of claim 14 wherein the drug is selected from a group comprising benzocaine, tetracaine, ropivacaine, lidocaine, water, saline, and combinations thereof.

19. The method of claim 14 wherein the user suffers from a condition selected from the group consisting of sphenopalatine neuralgia, trigeminal neuralgia, glossopharyngeal neuralgia, migraine, migraine with aural headache, migraine without aura headache, tension headache, cluster headache, chronic cluster headache, paroxysmal hemicranias, superior laryngeal neuralgia, facial pain, atypical facial pain, herpes zoster opthalmicus, vasomotor rhinitis, major depression, fibromyalgia, and combinations thereof.

20. The method of claim 14 wherein the method results in increased permeability of the drug through a blood brain barrier of the user.
